SEQUENCE LISTING (1 sequence)

SEQ ID NO: 1 - DNA, 22 bp, Artificial Sequence
  Synthetic Sequence
  misc_feature (3)..(3): 2′-deoxy-7-deazaguanosine
  misc_feature (7)..(7): 2′-deoxy-7-deazaguanosine
  misc_feature (11)..(12): Linked by is a glycerol linker
  misc_feature (11)..(11): 2′-deoxy-7-deazaguanosine
  misc_feature (12)..(12): 2′-deoxy-7-deazaguanosine
  misc_feature (16)..(16): 2′-deoxy-7-deazaguanosine
  misc_feature (20)..(20): 2′-deoxy-7-deazaguanosine
tcgaacgttcggcttgcaagct

(19) United States
(12) Patent Application Publication
(10) Pub. No.: US 2021/0317454 A1
(43) Pub. Date: Oct. 14, 2021

(54) USE OF A TLR9 AGONIST IN METHODS FOR TREATING COVID-19

(71) Applicant: Idera Pharmaceuticals, Inc., Exton, PA (US)

(72) Inventor: Srinivas CHUNDURU, Exton, PA (US)

(73) Assignee: Idera Pharmaceuticals, Inc., Exton, PA (US)

(21) Appl. No.: 17/226,866

(22) Filed: Apr. 9, 2021

Related U.S. Application Data
(60) Provisional application No. 63/008,305, filed on Apr. 10, 2020.
(60) Provisional application No. 63/115,264, filed on Nov. 18, 2020.

(51) Int. Cl. C12N 15/115; A61K 39/395; A61K 39/215; A61P 31/14

(52) U.S. Cl. CPC C12N 15/115; A61K 39/3955; A61K 2039/5252; A61P 31/14; C12N 2310/315; A61K 39/215

(57) ABSTRACT

The present invention is directed to use of tilsotolimod in the treatment of coronavirus infection, for example, SARS-CoV2, and COVID-19.

### CROSS-REFERENCE TO RELATED APPLICATIONS

This application claims the benefit of U.S. Provisional Patent Application Nos. 63/008,305 filed Apr. 10, 2020 entitled “Use of Tilsotolimod in Methods for Treating COVID-19”, and 63/115,264 filed Nov. 18, 2020 entitled “Use of a TLR9 Agonist in Methods for Treating COVID-19”, all of which are incorporated by reference herein in their entireties.

### FIELD OF THE INVENTION

The present invention relates to use of a TLR9 agonist, for example, tilsotolimod in methods for treating a patient infected with a coronavirus, for example, SARS-CoV2 coronavirus, or a patient suffering from a disease state caused by a coronavirus, for example, COVID-19, the disease caused by the SARS-CoV2 coronavirus.

### BACKGROUND OF THE INVENTION

Coronaviruses (CoVs) are enveloped viruses with a positive sense single-stranded RNA genome. Four coronavirus genera (alpha, beta, gamma, and delta) have been identified. Examples of beta-coronaviruses include MERS-CoV, SARS-CoV, HCov-OC42, and HCoV-HKU1, and now SARS-CoV2. Although its pathogenesis is not yet well understood, it is anticipated that similarities exist between SARS-CoV2 and other betacoronaviruses. It is hypothesized that SARS-CoV2 may escape the innate and adaptive immune systems in a manner similar to that of SARS-CoV and MERS-CoV.

COVID-19, for example, may initially present with mild, moderate, or severe illness. Patients may initially exhibit a mild disease, presenting with symptoms of an upper respiratory tract viral infection, including mild fever, cough, shortness of breath, sore throat, nasal congestion, malaise, headache, and/or muscle pain. Some patients will progress to moderate or severe pneumonia, and a fraction of patients will progress to Acute Respiratory Distress Syndrome (ARDS) or sepsis or septic shock, which can be life threatening. Some infected individuals lose the ability to smell and/or taste. Other symptoms may include body aches, chills, fatigue, nausea, and diarrhea. COVID-19 symptoms may lead to death, in part, due to complications such as pneumonia and/or organ failure. On the other hand, some people infected with SARS-CoV2 may be asymptomatic. The incubation period for SARS-CoV2 ranges from one to fourteen days, with a median period from five to six days.

Compositions and methods are needed for preventing, treating, relieving, or ameliorating symptoms of coronavirus infection, including COVID-19 and variants thereof, including treating or preventing severe illness from coronavirus infection.

### SUMMARY OF THE INVENTION

In an embodiment of the present invention, a method for treating, preventing, or ameliorating coronavirus infection and/or severe coronavirus disease in a patient is taught. The method comprises administering to the patient a pharmaceutical formulation comprising a therapeutically effective amount of a TLR9 agonist and a pharmaceutically acceptable carrier. In various embodiments, the coronavirus is SARS-CoV2. In one embodiment, the TLR9 agonist is tilsotolimod.

In some embodiments of the invention, a method for inducing an antiviral response to SARS-CoV2 virus in a patient is taught. The method comprises administering to the patient a pharmaceutical formulation comprising a therapeutically effective amount of a TLR9 agonist and a pharmaceutically acceptable carrier. In one embodiment, the TLR9 agonist is tilsotolimod. In an embodiment, the antiviral response has the effect selected from avoiding infection with SARS-CoV2 virus, avoiding developing COVID-19, reducing the period during which the patient can transmit the virus, reducing the severity of COVID-19 symptoms, or combinations thereof.

In an embodiment of the present invention, a method for treating a patient infected with SARS-CoV2 is taught. The method comprises administering to the patient a pharmaceutical formulation comprising a therapeutically effective amount of a TLR9 agonist and a pharmaceutically acceptable carrier. In one embodiment, the TLR9 agonist is tilsotolimod.

In some embodiments, a method for reducing the severity of COVID-19 symptoms is taught. The method comprises administering to said patient a pharmaceutical formulation comprising a therapeutically effective amount of a TLR9 agonist and a pharmaceutically acceptable carrier. In one embodiment, the TLR9 agonist is tilsotolimod.

In yet another embodiment, a method for reducing the period during which a patient infected with SARS-CoV2 virus can transmit the virus is taught. The method comprises administering to the patient a pharmaceutical formulation comprising a therapeutically effective amount of a TLR9 agonist and a pharmaceutically acceptable carrier. In one embodiment, the TLR9 agonist is tilsotolimod.

In another embodiment, any of the above methods of use further comprises administering to the patient a pharmaceutical formulation comprising a therapeutically effective amount of a TLR9 agonist in combination with another therapeutically effective treatment. In one embodiment, the TLR9 agonist is tilsotolimod.

In some embodiments, the therapeutically effective treatment comprises administering an anti-IL-6 antibody to the patient. In some embodiments, the anti-IL-6 antibody is selected from tocilizumab, siltuximab, sarilumab, olokizumab, elsilimomab, sirukumab, levilimab, MBS-945429, and CPSI-2364.

In some embodiments, the therapeutically effective treatment is a vaccine. In some embodiments, the vaccine is selected from a DNA or an mRNA vaccine (e.g., encoding one or more SARS-CoV2 proteins), a recombinant protein vaccine, a killed or inactivated virus vaccine, or a virus-like particle vaccine.

In some embodiments, the pharmaceutical formulation comprising a TLR9 agonist and the therapeutically effective treatment are co-formulated. In some embodiments, the pharmaceutical formulation comprising a TLR9 agonist is administered before, concurrently with, or after the therapeutically effective treatment. In one embodiment, the TLR9 agonist is tilsotolimod.

In some embodiments of the present invention, a method for treating a patient at risk for SARS-associated acute respiratory distress syndrome (ARDS) or COVID-19 is taught. The method comprises administering to the patient at risk a pharmaceutical formulation comprising a therapeutically effective amount of a TLR9 agonist and a pharmaceutical carrier. In one embodiment, the TLR9 agonist is tilsotolimod. In some embodiments, the patient at risk is positive for the SARS-CoV2 infection (e.g., by ELISA or RT-PCR), but is asymptomatic for COVID-19 or ARDS. In some embodiments, the patient at risk does not develop antibodies to the virus. In some embodiments, the patient at risk tests negative for virus antibodies. In some embodiments, the patient at risk has come into contact with a SARS-CoV2-infected individual. In some embodiments, the patient at risk is selected from a healthcare worker, a first responder, a physician, a nurse, a hospital worker, a paramedic, and an emergency medical technician. In some embodiments, the patient at risk shows early symptoms of COVID-19. In some embodiments, the early symptoms are selected from fever, cough, fatigue, chills, nausea, diarrhea, loss of the sense of taste, loss of the sense of smell, and shortness of breath.

In an embodiment of the invention, the pharmaceutical formulation comprising the TLR9 agonist, such as tilsotolimod, is administered by a route of administration selected from intravenous, intramuscular, intrathecal, subcutaneous, inhalation, nasal, nasal mist, nebulization, oral, sublingual, buccal, transdermal, and topical. In one embodiment, the route of administration is subcutaneous. In another embodiment, the route of administration is nasal, for example, a nasal mist. In another embodiment, the route of administration is inhalation. In yet another embodiment, the route of administration is nebulization.

In any of the embodiments according to the present invention, the TLR9 agonist (e.g., tilsotolimod) is administered subcutaneously at a dose of from about 0.1 mg to about 20 mg, or from about 0.2 mg to about 20 mg, or from about 0.3 mg to about 20 mg, or from about 0.4 mg to about 20 mg, or from about 0.5 mg to about 20 mg, or from about 0.6 mg to about 20 mg, or from about 0.7 mg to about 20 mg, or from about 0.8 mg to about 20 mg, or from about 0.9 mg to about 20 mg, or from about 1 mg to about 20 mg, or from about 2 mg to about 15 mg, or from about 3 mg to about 10 mg, or from about 4 mg to about 8 mg, or about 4 mg, or about 8 mg.

In any of the embodiments according to the present invention, the pharmaceutical formulation is administered before, concurrently with, or after another therapeutically effective treatment. In some embodiments, the therapeutically effective treatment is a vaccine. In some embodiments, the vaccine is selected from a DNA or an mRNA vaccine, a recombinant protein vaccine, a killed or inactivated virus vaccine, or a virus-like particle vaccine. In some embodiments, the pharmaceutical formulation and the therapeutically effective treatment are co-formulated.

### BRIEF DESCRIPTION OF THE DRAWINGS

FIG. 1 represents anti-viral activity against SARS-CoV2 infected Calu3 cells (as a function of TCID₅₀/mL) under three treatment conditions—24 hour pretreatment with tilsotolimod, co-incubation of tilsotolimod with SARS-CoV2 virus, and 24 hour pretreatment supernatant co-incubated with SARS-CoV2 virus in a fresh cell population. An untreated cell population, remdesivir, and pre-and post-infection hydroxychloroquine served as controls.

FIG. 2 shows that pre-treatment of mice with tilsotolimod provides a statistically significant reduction in infectious SARS-CoV2 titer (measured as a function of TCID₅₀/Lung). Asterisks indicate the level of significance using Student's t Test when compared to control (P<0.01).

FIG. 3 illustrates a robust infiltration of immune cells into the mediastinal lymph node in both pre and post-infection administration of tilsotolimod. B cells, CD4+ cells, CD8+ cells, granulocytes, macrophages, and dendritic cells were measured. Asterisks indicate the level of significance using Student's t Test when compared to the control group (*P<0.05, **P<0.01, and ***P<0.001).

### DETAILED DESCRIPTION OF THE INVENTION

Coronaviruses are a family of viruses that can cause varying respiratory illnesses such as the common cold, SARS, and MERS, at various degrees of illness. The SARS-CoV2 virus (also originally known as n-CoV-19), was reported in December 2019 as originating in Wuhan, China, and is a strain of coronavirus that causes coronavirus disease 2019, or COVID-19. Symptoms of SARS-CoV2 infection/COVID-19 include, fever, cough, shortness of breath, and difficulty breathing. Some infected individuals lost the ability to smell and/or taste. Other symptoms may include body aches, pneumonia, chills, fatigue, nausea, diarrhea, and cold-like symptoms such as a runny nose or a sore throat. COVID-19 symptoms can range from mild to severe, and may lead to death, in part, due to complications caused by COVID-19, such as pneumonia and/or organ failure. On the other hand, some people infected with SARS-CoV2 may be asymptomatic. The incubation period for SARS-CoV2 ranges from one to fourteen days, with a median period from five to six days.

SARS-CoV and MERS-CoV use multiple strategies to avoid immune response, including inhibition of Type I IFN pathways; induction of double membrane vesicles that lack PRRs and replicate in vesicles to avoid host detection of double stranded RNA; reduction of CD4+ and CD8+ T cells in peripheral blood of SARS-CoV2-infected patients; and downregulation of gene expression related to antigen presentation (WIC I and II). Both SARS-CoV and MERS-CoV have demonstrated multiple mechanisms of innate immune evasion, including escaping recognition by PRRs and downregulating endogenous interferon alpha.

SARS-like coronaviruses limit interferon (IFN) and antigen presentation machinery expression. The toll like receptor-9 (TLR-9) agonist, tilsotolimod, increases both interferon and antigen presentation machinery. Treatment of patients with SARS-CoV2 with tilsotolimod may therefore result in activation of TLR9, upregulation of the Type I IFN pathway (and specifically interferon alpha), and maturation of pDCs to express MHC class I, with subsequent activation and proliferation of CTLs to target cells infected with SARS-CoV-2. It is possible that treatment of outpatient confirmed individuals (those who test positive for SARS-CoV2) by priming the system may be more effective than giving tilsotolimod to patients who are already showing symptoms of respiratory distress.

Toll-like receptors (TLRs) are present on many cells of the immune system and are involved in the innate immune response. There are eleven TLR proteins (TLR1-TLR11) that recognize pathogen associated molecular patterns from bacteria, fungi, parasites, and viruses. TLRs are a key mechanism by which an immune response is mounted to foreign molecules and also provides a link between the innate and adaptive immune responses. TLR9 recognizes unmethylated CpG motifs in bacterial DNA, in some viruses, and in synthetic oligonucleotides. TLR9 agonists keep the immune system productively engaged to improve overall immune response.

One such TLR9 agonist is tilsotolimod. Tilsotolimod (IMO-2125) is a phosphorothioate oligonucleotide agonist of TLR9, a pattern recognition receptor (PRR) that is primarily expressed in B cells and plasmacytoid dendritic cells (pDCs), and has the sequence 5LTCG₁AACG₁TTCG₁-X-G₁CTTG₁CAAG₁CT-5′, wherein X is a glycerol linker and G₁ is 2′-deoxy-7-deazaguanosine (SEQ ID NO. 1). PRRs detect pathogen-associated molecular patterns (PAMPs) and subsequently can induce an innate immune response associated apthogenic antigens. Tilsotolimod stimulates pDCs and B cells through TLR9 to initiate a rapid innate immune response via activation of the Type I IFN pathway, primarily through production of large quantities of interferon (IFN) α, and subsequent maturation of dendritic cells to express MHC class I molecules. The combined activation of pDCs and the intrinsic cytokine/chemokine environment promotes a T-helper type 1(Th1) cellular response, production of cytotoxic T lymphocytes (CTLs), and the formation of antigen-specific memory T cells.

It may seem counterintuitive to use a TLR9 agonist such as tilsotolimod for treatment of SARS-CoV2 infection because tilsotolimod upregulates cytokine production. “Cytokine storm,” or a quick over-production or release of cytokines into the bloodstream to fight off infection is a common immunopathological event for SARS-CoV, MERS-CoV, and it appears to be also common for SARS-CoV2. However, in the tilsotolimod trials involving HCV, cytokine storm was not observed. Further a low dose of tilsotolimod may be all that is needed to decrease viral load and/or expedite viral clearance of SARS-CoV2 infection and/or treat symptoms of COVID-19. In addition, there are several similarities between SARS-CoV2 and solid tumors, including similar strategies to avoid an immune response as recited above, i.e., escaping PRRs, inhibiting the Type-I IFN pathway, downregulating antigen presentation, and decreasing the number of circulating T-cells.

At present, tilsotolimod is being evaluated via intratumoral injection in combination with ipilimumab vs. ipilimumab alone in a Phase 3 trial consisting of about 454 patients with advanced melanoma. Biopsies obtained from patients in a Phase 1/2 study demonstrated immune effects in the tumor microenvironment within 24 hours of receiving tilsotolimod. Activation of the Type I IFN pathway (induction of an IFNα gene signature) followed by maturation of dendritic cells (as measured by expression of MHC class I molecules) was observed. This resulted in systemic increases in CD8+ cytotoxic T lymphocytes (CTLs) with subsequent reduction of both injected and non-injected tumors. Additional trials in microsatellite stable colorectal cancer and head and neck cancer are also ongoing.

Subcutaneous administration of tilsotolimod has also been tested in HCV-infected patients with concurrent treatment with ribavirin. Tilsotolimod was administered subcutaneously at 0.08, 0.16 and 0.32 mg/kg once weekly, and 0.16 mg/kg twice weekly, for 4 weeks. Tilsotolimod was well tolerated with no discontinuations due to treatment-emergent adverse events. Viral load reductions were greater at the 0.16 mg/kg twice weekly tilsotolimod dose plus ribavirin compared with pegIFN-α2a plus ribavirin. However, none of the patients achieved rapid viral response.

In addition to subcutaneous and intratumoral modes of tilsotolimod administration, intranasal delivery in B16. F10 melanoma mouse models of colon carcinoma pulmonary metastasis has been evaluated. Intranasal administration of tilsotolimod induced a significant increase in tumor-specific CTLs in tracheobronchial lymph nodes at very low doses compared with subcutaneous administration, which required higher doses to elicit similar numbers of CTLs. Overall, intranasal treatment of tilsotolimod showed approximately 25 times more potent antitumor activity than subcutaneous treatment.

The present invention relates to treatment of various patient populations, including, for example, at risk patients, patients infected with SARS-CoV2 virus, asymptomatic patients, patients showing early symptoms of COVID-19 or ARDS, and patients already on another therapeutic treatment for COVID-19 or ARDS, with tilsotolimod, either through various routes of administration, including subcutaneous, intranasal, nebulized, or inhaled administration.

The SARS-CoV2 virus spreads relatively easily; thus “at risk” patients include any person working in a hospital, clinic, doctor's office, nursing home, or other medical environment, emergency personnel, such as paramedics, emergency medical technicians (EMTs), police officers, fire fighters, people who are infected with SARS-CoV2, people who are infected with SARS-CoV2 and are asymptomatic, people who test positive for the presence of SARS-CoV2 and show early symptoms, people who may not be infected but have other underlying health conditions, such as heart disease, respiratory issues (e.g., asthma or COPD), obesity, autoimmune conditions, history of stroke or embolism, or diabetes, people who are already on another therapeutically effective treatment for COVID-19, and the elderly.

Another method according to the present invention includes induction of an antiviral response in a patient infected with SARS-CoV2, whereby the antiviral response causes a patient to clear the virus without developing COVID-19, reduce the period during which the patient can transmit the virus, reduce the severity of COVID-19 symptoms, or combinations of such results. A reduction in the duration of asymptomatic transmission of the virus would greatly slow the spread of the virus, and would be a strong public health benefit. Further, reducing the severity of symptoms, and improving overall patient outcomes, provides clinical benefit to the patient and provides a public health benefit of preserving hospital resources.

In an embodiment of the present invention, any of these methods may be employed in combination with another therapeutically effective treatment for SARS-CoV2 infection and/or COVID-19.

Routes of administration for delivering tilsotolimod include, but are not limited to, subcutaneous, intravenous, intramuscular, or intrathecal injection; nasal deliveries including but not limited to aerosol, nasal mist, and nebulization; oral delivery, including by not limited to, sublingual, buccal, chewable, tablet, and capsule; inhaled delivery; subcutaneous delivery; topical delivery; and transdermal delivery. In various embodiments, tilsotolimod is administered subcutaneously.

Tilsotolimod may be formulated into a dosage form such as a solution, suspension, dispersion, emulsion, and the like, and/or may be provided in the form of sterile solid compositions (e.g. lyophilized composition), which can be dissolved or suspended in sterile injectable medium immediately before use. They may contain, for example, pharmaceutical carriers such as suspending or dispersing agents known in the art.

In various embodiments, tilsotolimod and is administered at from about 0.1 mg to about 30 mg, from about 0.2 mg to about 30 mg, from about 0.3 mg to about 30 mg, from about 0.4 mg to about 30 mg, from about 0.5 mg to about 30 mg, from about 0.6 mg to about 30 mg, from about 0.7 mg to about 30 mg, from about 0.8 mg, to about 30 mg, from about 0.9 mg to about 30 mg, from about 1 mg to about 30 mg, from about 1.5 mg to about 30 mg, from about 2 mg to about 30 mg, from about 2.5 mg to about 30 mg, from about 3 mg to about 30 mg, from about 3.5 mg to about 4 mg to about 64 mg per dose, or in some embodiments from about 8 mg to about 64 mg per dose, or from about 12 mg to about 64 mg per dose, or from about 16 mg to about 64 mg per dose, or from about 20 mg to about 64 mg per dose. In some embodiments, tilsotolimod is administered at from about 20 mg to about 48 mg per dose, or about 20 mg to about 40 mg per dose. For example, in various embodiments, tilsotolimod is administered at about 4 mg, or about 8 mg, or about 12 mg, or about 16 mg, or about 20 mg, or about 24 mg, or about 28 mg, or about 32 mg, or about 36 mg, or about 40 mg, or about 44 mg, or about 48 mg, or about 52 mg, or about 56 mg, or about 60 mg, or about 64 mg per dose. In some embodiments, tilsotolimod is administered at about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, or about 5 mg.

In various embodiments, tilsotolimod and is administered at from about 0.01 mg/kg to about 0.52 mg/kg. In some embodiments, tilsotolimod is administered at from about 0.05 mg/kg to about 0.40 mg/kg. In some embodiments, tilsotolimod is administered at from about 0.08 mg/kg to about 0.32 mg/kg. In some embodiments, tilsotolimod is administered at from about 0.10 mg/kg to about 0.30 mg/kg. In some embodiments, tilsotolimod is administered at from about 0.15 mg/kg to about 0.25 mg/kg. In some embodiments, tilsotolimod is administered at about 0.20 mg/kg. In some embodiments, tilsotolimod is administered at about 0.08 mg/kg. In some embodiments, tilsotolimod is administered at about 0.12 mg/kg. In some embodiments, tilsotolimod is administered at about 0.16 mg/kg. In some embodiments, tilsotolimod is administered at about 0.20 mg/kg. In some embodiments, tilsotolimod is administered at about 0.24 mg/kg. In some embodiments, tilsotolimod is administered at about 0.28 mg/kg. In some embodiments, tilsotolimod is administered at about 0.32 mg/kg. In some embodiments, tilsotolimod is administered at about 0.36 mg/kg. In some embodiments, tilsotolimod is administered at about 0.40 mg/kg. In some embodiments, tilsotolimod is administered at about 0.44 mg/kg. In some embodiments, tilsotolimod is administered at about 0.48 mg/kg. In some embodiments, tilsotolimod is administered at about 0.52 mg/kg.

In various embodiments, the tilsotolimod dose is administered from one to three times per day to about one to seven times per week. In some embodiments, the tilsotolimod dose is administered once weekly, twice weekly, three times weekly, four times weekly, five times weekly, six times weekly, or seven times weekly, or eight times weekly, or nine times weekly, or ten times weekly, or eleven times weekly, or twelve times weekly, or thirteen times weekly, or fourteen times weekly. In some embodiments, the tilsotolimod dose is administered once per day, twice per day, three times per day, or four times per day. In some embodiments, the tilsotolimod dose is delivered once or multiple times over the course of about one week, two weeks, three weeks, four weeks, five weeks, or six weeks. In some embodiments, the tilsotolimod dose is delivered once per day for a period of one week. In some embodiments, the tilsotolimod dose is delivered twice weekly for a period of four weeks.

In various embodiments, about 3 to about 12 doses of tilsotolimod are administered (e.g. about 3 doses, or about 4 doses, or about 5 doses, or about 6 doses, or about 7 doses, or about 8 doses, or about 9 doses, or about 10 doses, or about 11 doses, or about 12 doses). In various embodiments, about 4 to about 20 doses are administered over one week, two weeks, three weeks, four weeks, or five weeks. In some embodiments, about 1 dose, or about 2 doses, or about 3 doses, or about 4 doses, or about 5 doses, or about 6 doses, or about 7 doses, or about 8 doses, or about 9 doses, or about 10 doses, or about 11 doses, or about 12 doses, or about 13 doses, or about 14 doses, or about 15 doses, or about 16 doses, or about 17 doses, or about 18 doses, or about 19 doses, or about 20 doses, are administered over one week, two weeks, three weeks, four weeks, or five weeks.

In some embodiments of the present invention, tilsotolimod is administered before, concurrently with, or after administration of another therapeutically effective treatment. In some embodiments, the therapeutically effective treatment is an anti-IL-6 antibody. In some embodiments, the anti-IL-6 antibody is selected from tocilizumab, siltuximab, sarilumab, olokizumab, elsilimomab, sirukumab, levilimab, MBS-945429, and CPSI-2364. In some embodiments, the anti-IL-6 antibody is administered according to its labeled, approved use.

In some embodiments, the therapeutically effective treatment is an interferon, for example, a beta-1b, an alpha-n1, and alpha-n3, or a human leukocyte interferon alpha.

In some embodiments of the present invention, the therapeutically effective treatment is hydroxychloroquine, chloroquine, chloroquine phosphate, or a derivative or precursor thereof. In some embodiments, the therapeutically effective treatment is favilar. In some embodiments, the therapeutically effective treatment is remdesivir. In some embodiments, the therapeutically effective treatment is an anti-viral. In some embodiments, other therapeutically effective treatments include treatments useful in treating inflammation. In some embodiments, other therapeutically effective treatments include treatments currently used or future developed for treating respiratory disease, difficulty breathing, and/or pneumonia. In some embodiments, other therapeutically effective treatments include any treatment currently used or being developed for treatment of COVID-19.

In some embodiments, the therapeutically effective treatment is a vaccine, for example, an mRNA vaccine, a recombinant protein vaccine, a killed or inactivated virus vaccine, or a virus-like particle vaccine. In some embodiments, the vaccine is administered according to its labeled, approved use. In some embodiments, the vaccine is a current or future vaccine developed specifically for COVID-19. In some embodiments, the vaccine is in clinical trials. In some embodiments, the vaccine is approved for use for COVID-19.

In some embodiments, the pharmaceutical formulation comprising tilsotolimod and the therapeutically effective treatment are co-formulated and delivered together. In some embodiments, the tilsotolimod is administered as an adjuvant therapy to a vaccine.

In some embodiments, the therapeutically effective treatment is a steroid. In some embodiments, the steroid is selected from corticosteroids, glucocorticoids, prednisone, prednisolone, methylprednisone, methylprednisolone, cortisone, hydrocortisone, triamcinolone, budesonide, dexamethasone, deflazacort, fludrocortisone, and bethamethasone. In various embodiments, the steroid is dexamethasone.

In some embodiments, the steroid is administered once daily, twice daily, or three times daily during the course of the treatment period, which is one day up to fourteen days. In some embodiments, the treatment period is one day, two days, three days, four days, five days, six days, seven days, eight days, nine days, ten days, eleven days, twelve days, thirteen days, or fourteen days. In some embodiments, the treatment period is one week. In some embodiments, the treatment period is two weeks, three weeks or four weeks.

In some embodiments, the treatment period is seven days and the tilsotolimod is administered once every other day beginning on day one at a dose of about 4 mg to about 8 mg, or about 1 mg, 1.5 mg, 2 mg, 2.5 mg 3 mg, 3.5 mg 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg. In one embodiment, the tilsotolimod is administered subcutaneously. In another embodiment, the tilsotolimod is administered intranasally. In one embodiment, prednisolone is administered at a dose of about 2.5 mg/kg on a daily basis for a treatment period of seven days and tilsotolimod is administered subcutaneously once every other day beginning on day 1 of the treatment period at a dose of about 4 mg. In one embodiment, prednisolone is administered at a dose of about 2.5 mg/kg on a daily basis for a treatment period of seven days and tilsotolimod is administered subcutaneously once every other day beginning on day 1 of the treatment period at a dose of about 5 mg. In one embodiment, prednisolone is administered at a dose of about 2.5 mg/kg on a daily basis for a treatment period of seven days and tilsotolimod is administered subcutaneously once every other day beginning on day 1 of the treatment period at a dose of about 6 mg. In one embodiment, prednisolone is administered at a dose of about 2.5 mg/kg on a daily basis for a treatment period of seven days and tilsotolimod is administered subcutaneously once every other day beginning on day 1 of the treatment period at a dose of about 7 mg. In one embodiment, prednisolone is administered at a dose of about 2.5 mg/kg on a daily basis for a treatment period of seven days and tilsotolimod is administered subcutaneously once every other day beginning on day 1 of the treatment period at a dose of about 8 mg.

In one embodiment, prednisolone is administered at a dose of about 2.5 mg/kg on a daily basis for a treatment period of seven days and tilsotolimod is administered intranasally once every other day beginning on day 1 of the treatment period at a dose of about 4 mg. In one embodiment, prednisolone is administered at a dose of about 2.5 mg/kg on a daily basis for a treatment period of seven days and tilsotolimod is administered intranasally once every other day beginning on day 1 of the treatment period at a dose of about 5 mg. In one embodiment, prednisolone is administered at a dose of about 2.5 mg/kg on a daily basis for a treatment period of seven days and tilsotolimod is administered intranasally once every other day beginning on day 1 of the treatment period at a dose of about 6 mg. In one embodiment, prednisolone is administered at a dose of about 2.5 mg/kg on a daily basis for a treatment period of seven days and tilsotolimod is administered intranasally once every other day beginning on day 1 of the treatment period at a dose of about 7 mg. In one embodiment, prednisolone is administered at a dose of about 2.5 mg/kg on a daily basis for a treatment period of seven days and tilsotolimod is administered intranasally once every other day beginning on day 1 of the treatment period at a dose of about 8 mg.

The present invention contemplates that tilsotolimod is formulated into a pharmaceutical composition suitable for various routes of administration, including, but not limited to, subcutaneous administration and intranasal administration. Pharmaceutically acceptable carriers are inert, biocompatible solvents, suspending agents, or other vehicles for delivering tilsotolimod, and include, but are not limited to, buffers, stabilizers, excipients, diluents, and liposomal suspensions.

Pharmaceutically acceptable carriers of tilsotolimod include water, saline, buffering agents, phosphate buffers, Ringer's solution, sugar solution (such as dextrose) glycols, glycerol, oils, alkyl benzoates, aryl benzoates, aralkyl benzoates, triacetin, dimethyl sulfoxide (DMSO), N-methyl-2-pyrrolidone (NMP), alkanes, cyclic alkanes, chlorinated alkanes, fluorinated alkanes, perfluorinated alkanes and mixtures thereof.

In one embodiment, the subcutaneous injection is formulated with saline.

While preferred embodiments of the invention are shown and described herein, such embodiments are provided by way of example only and are not intended to otherwise limit the scope of the invention. Various alternatives to the described embodiments of the invention may be employed in practicing the invention. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents incorporated herein by reference. All the features disclosed in the specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Subcutaneous Administration of Tilsotolimod to Treat COVID-19

The objective of this study is to assess safety and efficacy of tilsotolimod injections by measuring treatment emergent adverse events and injection reaction. Duration of symptoms of COVID-19, such as fever, dyspnea, cough, oxygen saturation levels, and hypoxemia, duration of required breathing assistance with a ventilator, survival rate, and radiological chest imaging (CT/Xray) are measured. Viral load over time in a blood or nasopharyngeal fluids is also measured. Further, the potential biomarkers, including cytokines, are investigated, as well as incidence of anti-tilsotolimod antibodies. These are correlated with changes in viral load.

It is hypothesized that the use of tilsotolimod in patients who test positive for SARS-CoV2 infection will be well tolerated, will shorten the duration of the SARS-CoV2 viral infection, and will provide clinical benefit. Patients who are hospitalized with ARDS are excluded from this study.

Up to 40 patients with a confirmed diagnosis of COVID-19 infection receive one subcutaneous injection of tilsotolimod at a dose of 4 mg or up to 8 mg on days 1, 3, 5, and 7 of the treatment period.

Plasma samples and nasopharyngeal swabs from treated patients are collected on Day 0 of the treatment period, each day at roughly the same time during the treatment period, at the end of the treatment period, and for seven to fourteen days after the treatment period, and are assessed for changes in cytokines and viral load. A physical examination, vital signs, various blood chemistry analysis, blood gas analysis, oxygen saturation levels, and serial chest imaging are taken on Day 0 and Day 7 of the treatment period, and on Day 14 (Day 7 following the end of the treatment period to measure changes in severity and duration of symptoms. Fever is measured daily throughout the treatment period and for an additional seven to fourteen days following the end of the treatment period. Additionally, blood samples for analysis of tilsotolimod concentrations are collected at defined time points (e.g., at time 0, 4 hours, 24 hours, and then daily until up to fourteen days after the end of the treatment period). A physical examination is performed daily, and daily progress notes are collected and reviewed for each patient.

### Example 2: Intranasal Administration of Tilsotolimod to Treat COVID-19

This study is similar to Example 1 in terms of the purpose of the study and what parameters are assessed. Patients who are hospitalized with ARDS are excluded from this study.

Up to 40 patients with a confirmed diagnosis of COVID-19 infection receive one intranasal instillation of tilsotolimod at a dose of 4 mg or up to 8 mg on days 1, 3, 5, and 7 of the treatment period.

Plasma samples and nasopharyngeal swabs from treated patients are collected on Day 0 of the treatment period, each day at roughly the same time during the treatment period, at the end of the treatment period, and for seven to fourteen days after the treatment period, and are assessed for changes in cytokines and viral load. A physical examination, vital signs, various blood chemistry analysis, blood gas analysis, oxygen saturation levels, and serial chest imaging are taken on Day 0 and Day 7 of the treatment period, and on Day 14 (Day 7 following the end of the treatment period to measure changes in severity and duration of symptoms. Fever is measured daily throughout the treatment period and for an additional seven to fourteen days following the end of the treatment period. Additionally, blood samples for analysis of tilsotolimod concentrations are collected at defined time points (e.g., at time 0, 4 hours, 24 hours, and then daily until up to fourteen days after the end of the treatment period). A physical examination is performed daily, and daily progress notes are collected and reviewed for each patient.

### Example 3: Tilsotolimod of SARS-CoV-2 Replication in Calu3 Cells

It was hypothesized that treatment of outpatient-confirmed SARS-CoV-2 positive individuals with tilsotolimod will inhibit disease progression. To test this hypothesis, studies were completed in human pulmonary epithelial cells (Calu3 cells) as well as in an animal model (Example 4, below).

Calu3 cells were cultured in DMEM+10% fetal bovine serum (FBS) (ATCC). Serial 1/3 log dilutions of tilsotolimod (final concentration of 100 μM to 0.005 μM) were prepared separately in HTB55 medium with 10% FBS. Remdesivir and hydroxychloroquine, each at 10 μM were used as controls. Three treatment conditions were tested as follows:
1. Tilsotolimod 24 Hour Pre-Treatment: Cells were incubated with media containing tilsotolimod for 24 hours , then infected with SARS-CoV2 for an additional 48 hours.
2. Tilsotolimod Co-Treatment with SARS-CoV2: Cells were incubated with media containing tilsotolimod simultaneously with SARS-CoV-2 for 48 hours.
3. Tilsotolimod 24 Hour Pre-Treatment, Supernatant Only: 20 microliters of supernatant from treatment condition #1 was added to fresh cells, which were then infected with SARS-CoV2 and incubated for an additional 48 hours.

Cells were infected with SARS-CoV2 (multiplicity of infection of 0.1) and incubated at 37° C. for 48 hours. Supernatant was then collected and serially passed ten times at 7× dilutions (highest dilution about 2.5×10⁻⁸) across Vero cells for calculation of TCID₅₀/mL by cytopathic effect (CPE) assay. Vero monolayers were incubated for 5 days to allow CPE to develop. CPE was scored (positive or negative) and TCID₅₀/mL were calculated for each treatment condition at all dilutions, using the Spearman and Karber test.

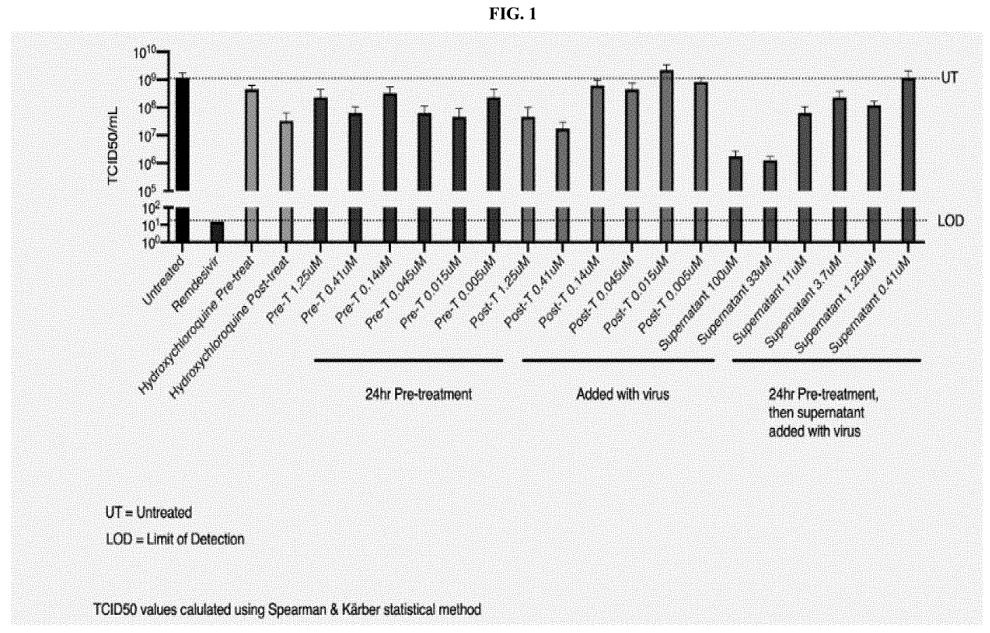

Results. Tilsotolimod antiviral activity for each of the three treatment conditions at multiple serial dilutions is shown in FIG. 1. Tilsotolimod induced toxicity above 3 μM, so these dilutions were excluded from analyses of conditions 1 and 2. The greatest antiviral activity was observed under treatment condition 3, in which supernatant from Calu3 cells pretreated with tilsotolimod was added to fresh cells that were subsequently infected with SAR-CoV2; supernatant from the 1:10 serial dilution (33 μM) exhibited the greatest antiviral activity, with an approximate 3-log reduction in TCID5o. The results from condition 3 demonstrate that the supernatant from tilsotolimod pre-treatment reduces infectivity, suggesting that tilsotolimod treatment results in secretion of a soluble factor that inhibits SARS-CoV2 replication. The most effective dilution for treatment condition 2 (tilsotolimod co-treatment with SARS-CoV2) was 0.41 μM, with an approximate 1-log reduction in TCID₅₀. None of the serial dilutions for treatment condition 1 (tilsotolimod 24 h pre-treatment) resulted in significant antiviral activity compared with controls.

### Example 4: Tilsotolimod Inhibition of SARS-CoV2 Replication in Animal Model

A novel strain of SARS-CoV2 that infects normal mice was obtained from Professor Marc Pellegrini's laboratory at the Walter and Eliza Hall Institute, Australia. The strain has a mutation in the ACE2 binding domain, thus bypassing the requirement for using ACE2 transgenic mice. A total of 8 BL6 mice were tested in each test group and compared to a control group of 8 BL6 mice. The first test group received an intranasal dose of tilsotolimod at 2.5 mg/kg one day prior to exposure to the SARS-CoV2 virus. The second test group received an intranasal dose of tilsotolimod at 2.5 mg/kg one day after exposure to the SARS-CoV2 virus. The control group received intranasal vehicle only. Three days post-infection, lungs were harvested, TCID₅₀ was measured, and flow cytometric analysis of mediastinal lymph nodes was performed.

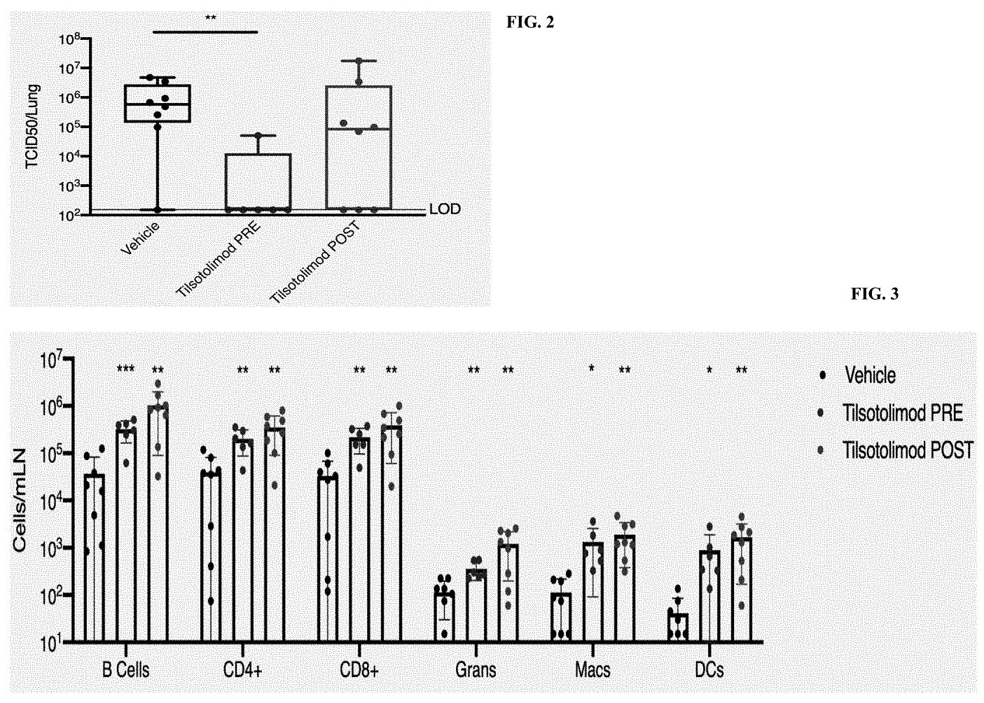

FIG. 2 illustrates that pre-treatment with tilsotolimod resulted in a statistically significant reduction in infection of mice with SARS-CoV2 as compared with the vehicle control. The post-treatment group also reduced infection, although not statistically significant in this small sample. In the pre-treatment group, one mouse died, and two showed signs of reduced mobility. No toxicity was observed in either the post-treatment group or the control group.

FIG. 3 illustrates robust changes in the number of immune cells in the mediastinal lymph nodes in both treatment groups. Statistical significance between both treatment groups against the control group is shown at varying p values.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for treating COVID-19 in a patient, the method comprising administering to the patient a pharmaceutical formulation comprising a therapeutically effective amount of a TLR9 agonist and a pharmaceutically acceptable carrier.

2. The method of claim 1, wherein the TLR9 agonist is tilsotolimod.

3. The method of claim 1, wherein the pharmaceutical formulation is administered by a route of administration selected from intravenous, intramuscular, intrathecal, subcutaneous, inhalation, nasal, nasal mist, nebulization, oral, sublingual, buccal, transdermal, and topical.

4. The method of claim 1, wherein the pharmaceutical formulation is administered at a dose of from about 1 mg to about 10 mg.

5. The method of claim 1, wherein the pharmaceutical formulation is administered at a dose of about 1 mg to about 20 mg.

6. A method for inducing an antiviral response to SARS-CoV2 virus in a patient, the method comprising administering to said patient a pharmaceutical formulation comprising a therapeutically effective amount of a TLR9 agonist and a pharmaceutically acceptable carrier.

7. The method of claim 6, wherein the TLR9 agonist is tilsotolimod.

8. The method of claim 6, wherein the antiviral response has the effect selected from avoiding infection with SARS-CoV2 virus, avoiding developing COVID-19, reducing the period during which the patient can transmit the virus, reducing the severity of COVID-19 symptoms, or combinations thereof.

9. The method of claim 6, wherein the patient tests positive for SARS-CoV2 infection, but is asymptomatic for COVID-19.

10. The method of claim 6, wherein the patient shows early symptoms of COVID-19.

11. The method of claim 10, wherein the symptoms are selected from fever, cough, fatigue, chills, nausea, diarrhea, loss of the sense of taste, loss of the sense of smell, and respiratory distress.

12. A method for treating a patient suffering from COVID-19, the method comprising administering to said patient a pharmaceutical formulation comprising a therapeutically effective amount of a TLR9 agonist in combination with another therapeutically effective treatment.

13. The method of claim 12, wherein the TLR9 agonist is tilsotolimod.

14. The method of claim 12, wherein the therapeutically effective treatment comprises anti-IL-6 antibody.

15. The method of claim 14, wherein the anti-IL-6 antibody is selected from tocilizumab, siltuximab, sarilumab, olokizumab, elsilimomab, sirukumab, levilimab, MBS-945429, and CPSI-2364.

16. The method of claim 12, wherein the therapeutically effective treatment is a vaccine.

17. The method of claim 16, wherein the vaccine is selected from an mRNA vaccine, a recombinant protein vaccine, a killed or inactivated virus vaccine, or a virus-like particle vaccine.

18. The method of claim 12, wherein the pharmaceutical formulation and the therapeutically effective treatment are co-formulated.

19. The method of claim 12, wherein the pharmaceutical formulation is administered before, concurrently with, or after the therapeutically effective treatment.

20. The method of claim 12, wherein the patient suffering from COVID-19 has one or more symptoms selected from fever, cough, fatigue, chills, nausea, diarrhea, loss of the sense of taste, loss of the sense of smell, and respiratory distress.
